(19) United States
(12) Patent Application Publication
(10) Pub. No.: US 2019/0387966 A1
(43) Pub. Date: Dec. 26, 2019

(54) BLOOD-VESSEL OPTICAL DEVICE AND BLOOD-VESSEL OPTICAL SYSTEM

(71) Applicant: OLYMPUS CORPORATION, Tokyo (JP)

(72) Inventors: Hiroshi TSURUTA, Kanagawa (JP); Takashi YASUMI, Tokyo (JP); Yasuaki KASAI, Saitama (JP); Hirokazu YOKOTA, Tokyo (JP)

(73) Assignee: OLYMPUS CORPORATION, Tokyo (JP)

(21) Appl. No.: 16/563,062

(22) Filed: Sep. 6, 2019

Related U.S. Application Data
(63) Continuation of application No. PCT/JP2017/011028, filed on Mar. 17, 2017.

(51) Int. Cl. A61B 1/313; A61B 6/00; A61B 1/07; A61B 1/00

(52) U.S. Cl. CPC A61B 1/3137; A61B 1/00172; A61B 1/07; A61B 6/481

(57) ABSTRACT

A blood-vessel optical device is provided with: a sheath provided with a lumen penetrating therethrough, and a dovetail groove disposed so as to be parallel to the lumen; and an optical member accommodated in the lumen and including at least one of an illuminating portion guiding illumination light to an imaging subject and a light detecting portion guiding return light from the imaging subject, wherein the groove has a cross-sectional shape configured to accommodate an elongated member so as to be movable, and has an opening that has a width smaller than a diameter of the elongated member and from which a portion of the accommodated elongated member protrudes to the outside, and wherein the sheath is configured such that an external dimension in a direction orthogonal to an arrangement direction in which the lumen and the groove are arranged is smaller than an external dimension in the arrangement direction.

### CROSS-REFERENCE TO RELATED APPLICATIONS

This is a continuation of International Application PCT/JP2017/011028 which is hereby incorporated by reference herein in its entirety.

### TECHNICAL FIELD

The present invention relates to a blood-vessel optical device and a blood-vessel optical system.

### BACKGROUND ART

There is a known blood-vessel optical system for observing a diseased site in a blood vessel (for example, see Patent Literature 1).

This blood-vessel optical system is provided with: a lumen through which an optical fiber that transmits return light from the blood vessel passes; and a tubular sheath provided with a lumen through which a guide wire passes.

### CITATION LIST

### Patent Literature

### {PTL 1} Japanese Translation of PCT International Application, Publication No. 2012-504019

### SUMMARY OF INVENTION

An aspect of the present invention is a blood-vessel optical device provided with: an elongated sheath provided with a lumen that penetrates therethrough in a longitudinal direction, and a dovetail groove that is disposed so as to be parallel to the lumen along the longitudinal direction; and an optical member that is accommodated in the lumen of the sheath, and that includes at least one of an illuminating portion that guides illumination light to an imaging subject in a blood vessel from outside of a body and a light detecting portion that guides return light from the imaging subject to outside of the body, wherein the dovetail groove has a cross-sectional shape configured to accommodate an elongated member having a circular cross section so as to be movable in the longitudinal direction, and has, on a side opposite to the lumen, an opening that has a width smaller than a diameter of the elongated member and from which a portion of the accommodated elongated member, in a circumferential direction, protrudes to the outside, and wherein the sheath is configured such that an external dimension in a direction orthogonal to an arrangement direction in which the lumen and the dovetail groove are arranged is smaller than an external dimension in the arrangement direction.

### BRIEF DESCRIPTION OF DRAWINGS

FIG. 1 is an overall diagram showing a blood-vessel optical system according to an embodiment of the present invention.

FIG. 2 is a longitudinal cross-sectional view showing a blood-vessel optical device according to an embodiment of the present invention.

FIG. 3 is a cross-sectional view showing the blood-vessel optical device in FIG. 2.

FIG. 4 is a cross-sectional view showing the blood-vessel optical system in FIG. 2.

FIG. 5 is a cross-sectional view showing a first modification of the blood-vessel optical device in FIG. 2.

FIG. 6 is a cross-sectional view showing a second modification of the blood-vessel optical device in FIG. 2.

FIG. 7 is a cross-sectional view showing a third modification of the blood-vessel optical device in FIG. 2.

FIG. 8 is a cross-sectional view showing a fourth modification of the blood-vessel optical device in FIG. 2.

FIG. 9 is a cross-sectional view showing a fifth modification of the blood-vessel optical device in FIG. 2.

FIG. 10 is a cross-sectional view showing a sixth modification of the blood-vessel optical device in FIG. 2.

FIG. 11 is a cross-sectional view showing a seventh modification of the blood-vessel optical device in FIG. 2.

### DESCRIPTION OF EMBODIMENT

A blood-vessel optical device 2 and a blood-vessel optical system 1 according to an embodiment of the present invention will be described below with reference to the drawings.

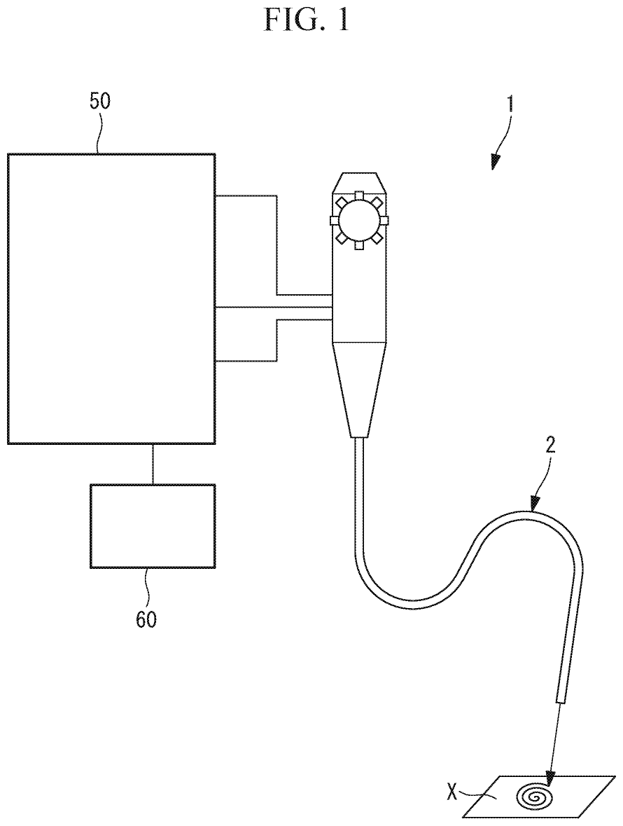

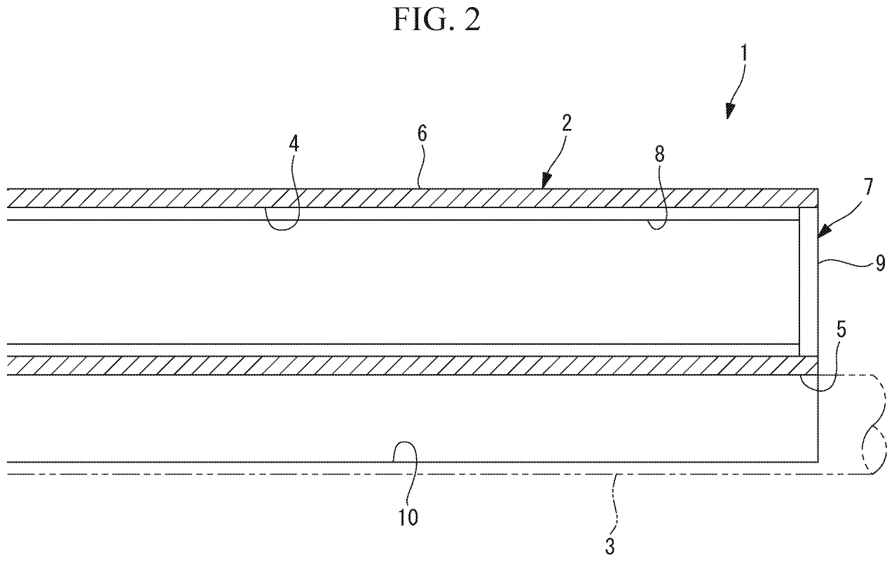

As shown in FIGS. 1 and 2, the blood-vessel optical system 1 according to this embodiment is provided with: the blood-vessel optical device 2 according to this embodiment; a guide wire (elongated member) 3 having a circular cross section; a control device 50 that controls the blood-vessel optical device 2; and a monitor 60.

The blood-vessel optical system 1 is an optical system that scans illumination light on an (imaging subject) X in a blood vessel along a spiral scanning trajectory, and that acquires an image of the diseased site X.

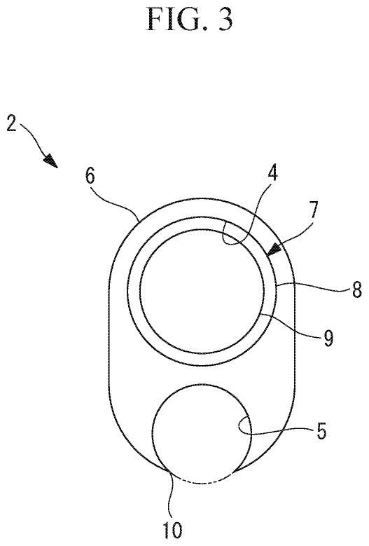

As shown in FIGS. 2 and 3, the blood-vessel optical device 2 according to this embodiment is provided with: an elongated tubular sheath 6 that is formed of a flexible material and that is provided with a lumen 4 and a dovetail groove 5 that penetrate therethrough in a longitudinal direction; and an optical member 7 that is accommodated in the lumen 4 of the sheath 6.

The optical member 7 is provided with: a light detecting portion 8 that is provided with a plurality of optical fibers (an optical fiber bundle) arranged in a circumferential direction of a tube having a circular cross section; and an illuminating portion 9 that is disposed on the inner side of the light detecting portion 8 and that is provided with, for example, an optical fiber scanner (not shown).

The sheath 6 is composed of, for example, a resin containing an X-ray contrast medium.

As shown in FIG. 3, the sheath 6 has a substantially oval cross-sectional shape having a minor axis and a major axis, and the lumen 4 and the dovetail groove 5 are arranged along the major-axis direction thereof. The lumen 4 provided in the sheath 6 has, for example, a circular cross-sectional shape having an inner diameter large enough to accommodate the optical member 7 having a circular cross section.

The dovetail groove 5 provided in the sheath 6 has an opening 10 that opens, with a prescribed opening width, toward the outside on the side opposite to the lumen 4, and has, on the inner side thereof, a groove width larger than the opening width of the opening 10.

More specifically, the dovetail groove 5 has an inner surface having a circular cross section, and the opening 10 that serves as an opening between the interior of the dovetail groove 5 and the outside is provided at the position at which the inner surface intersects with an outer surface of the sheath 6.

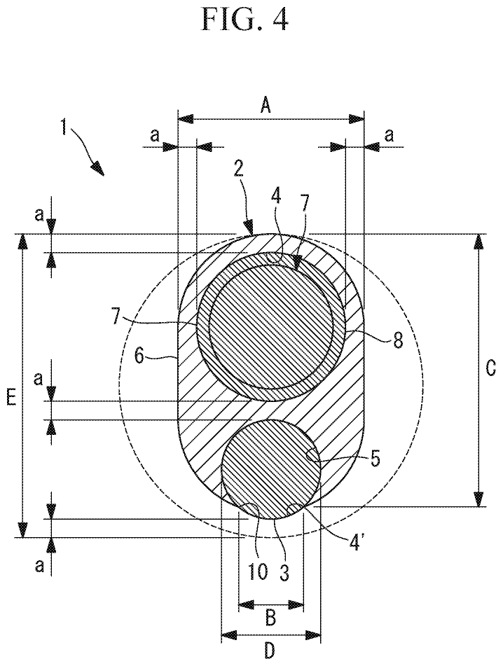

Here, as shown in FIG. 4,

B<D

is satisfied, where D is the diameter of the circle forming the cross-sectional shape of the dovetail groove 5, and B is the opening width of the opening 10.

The diameter D is set to be slightly larger than the outer diameter of the guide wire 3. With this configuration, the guide wire 3 is accommodated in the dovetail groove 5 so as to be movable in the longitudinal direction.

In addition, in this embodiment, the cross-sectional shape of the sheath 6 is set so that a width A in the minor-axis direction is smaller than a width C in the major-axis direction. Furthermore, in this embodiment, a minimum required thickness a is ensured between the outer surface of the sheath 6 and the lumen 4, and between the lumen 4 and the dovetail groove 5. The broken line shown in FIG. 4 is a circle of an imaginary sheath having a diameter E obtained by adding the minimum required thickness a to the dimension, in the major-axis direction, from the outer surface of the sheath 6 to an outer surface of the guide wire 3 exposed from the opening 10.

The operation of the thus-configured blood-vessel optical system 1 and blood-vessel optical device 2 according to this embodiment will be described below.

To perform observation of a diseased site X in the interior of a blood vessel by using the blood-vessel optical system 1 according to this embodiment, the guide wire 3, which is formed of a radiopaque material, is inserted into the blood vessel and introduced up to the diseased site X under X-ray observation using an X-ray observation apparatus.

After the guide wire 3 is disposed, the proximal end of the guide wire 3 is inserted into the dovetail groove 5 from the distal end of the sheath 6, the sheath 6 accommodating the optical member 7 in the lumen 4 is inserted into the blood vessel while being guided with the guide wire 3, and the distal end of the sheath 6 is disposed in the vicinity of the diseased site X.

In this case, although the guide wire 3 inserted into the dovetail groove 5 is accommodated in a state in which a portion thereof in the circumferential direction protrudes from the opening 10 of the dovetail groove 5, because the external dimension of the guide wire 3 is larger than the opening 10 of the dovetail groove 5, the guide wire 3 can smoothly guide the sheath 6 up to the diseased site X without falling out from the opening 10 of the dovetail groove 5.

In addition, in this case, as compared with the imaginary sheath indicated by the broken line in FIG. 4 (a sheath having a structure in which a lumen 4′ is formed by the opening 10 of the dovetail groove 5 being closed with the minimum required thickness a, and the guide wire 3 is inserted into the lumen 4′), the guide wire 3 is inserted into the dovetail groove 5, thereby eliminating the need to provide, in the sheath 6, the minimum required thickness a outside the guide wire 3, making it possible to reduce the width C in the major-axis direction by the corresponding amount. Furthermore, in this embodiment, it is possible to achieve a reduction in diameter over the entire sheath 6 by setting the width A in the minor-axis direction to be smaller than the width C in the major-axis direction. With this configuration, there is an advantage in that it is possible to easily introduce the blood-vessel optical device 2 into a thinner blood vessel.

In addition, in this embodiment, the sheath 6 is composed of a material containing an X-ray contrast medium; thus, it is possible to clearly identify the distal end position of the sheath 6 under X-ray observation, and the distal end of the sheath 6 can be accurately disposed in the vicinity of the diseased site X.

In this state, the inside of the blood vessel is flushed, and subsequently, observation is commenced in the blood vessel by means of the optical member 7.

As described above, with the blood-vessel optical device 2 and the blood-vessel optical system 1 according to this embodiment, the dovetail groove 5 for guiding the guide wire 3 is employed, and the width A in the minor-axis direction is set to be smaller than the width C in the major-axis direction; thus, there is an advantage in that it is possible to reduce the dimensions of the cross-sectional shape and to facilitate introduction of the sheath 6 into a thinner blood vessel. In addition, there is also an advantage in that it is possible to ensure a sufficient space outside the sheath 6 by the amount corresponding to the reduction in the dimensions of the cross-sectional shape, and to ensure an introduction path for another treatment tool or a flushing fluid by utilizing that space.

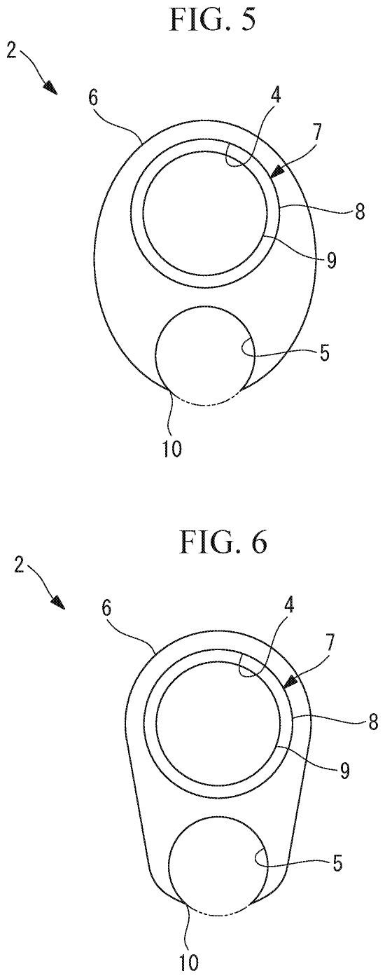

Note that, although the sheath 6 having an oval cross-sectional shape has been described in this embodiment, alternatively, the sheath 6 may be configured to have an elliptical cross-sectional shape as shown in FIG. 5, or as shown in FIG. 6, a shape asymmetrical in the major-axis direction may be employed in accordance with the outer diameter of the optical member 7 and the outer diameter of the guide wire 3.

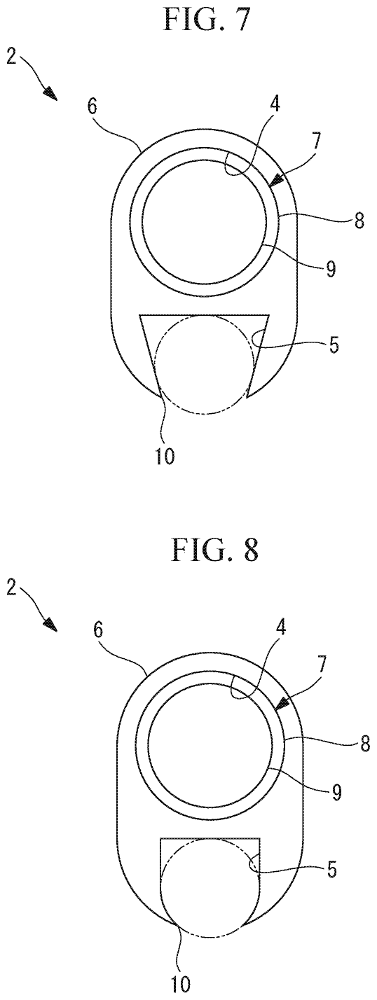

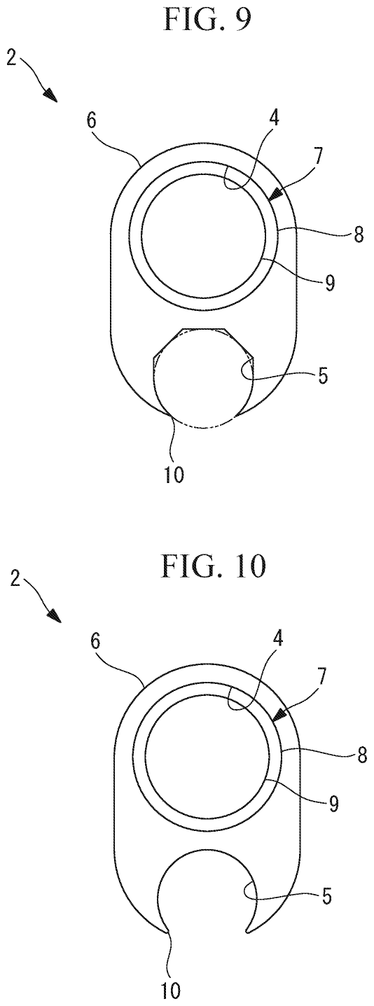

In addition, although the dovetail groove 5 having a circular cross-sectional shape has been described in this embodiment, alternatively, as long as the outer surface of the guide wire 3 is inscribed in the inner surface shape of the dovetail groove 5, as shown in FIG. 7, the dovetail groove 5 may have a shape in which the groove width gradually increases from the opening 10 toward the inside. Alternatively, as shown in FIG. 8, the dovetail groove 5 may have a shape in which the groove width in the vicinity of the bottom surface thereof is substantially equal to the diameter of the guide wire 3. Furthermore, as shown in FIG. 9, a dovetail groove 5 having a polygonal cross-sectional shape circumscribing the cross section of the guide wire 3 may be employed.

In addition, at the boundary where the inner surface of the dovetail groove 5 intersects with the outer surface of the sheath 6, in the cross section, ridges are formed on both sides of the opening 10 along the longitudinal direction of the sheath 6, and the ridges become thin and sharp when the angle between the inner surface of the dovetail groove 5 and the outer surface of the sheath 6 becomes small. In this case, as shown in FIG. 10, it is preferable that rounding-processing be performed so that edges are not formed on the ridges. The rounding-processing may be performed during or after molding of the sheath 6.

In addition, although the optical member 7 provided with the light detecting portion 8 that is constituted of a plurality of optical fibers arranged in the circumferential direction around the illuminating portion 9 provided with an optical fiber scanner has been illustrated as an example in this embodiment, conversely, an illuminating portion 9 that is constituted of illumination optical fibers arranged in the circumferential direction around the light detecting portion 8 may be provided.

In addition, it is permissible that only the illuminating portion 9 or only the light detecting portion 8 is inserted into the lumen 4.

In addition, although the guide wire 3 has been illustrated as an example of the elongated member, after the sheath 6 is introduced into a blood vessel by using the guide wire 3 as a guide, the guide wire 3 may be removed from the dovetail groove 5, and another treatment tool may be subsequently introduced by using the dovetail groove 5 as a guide.

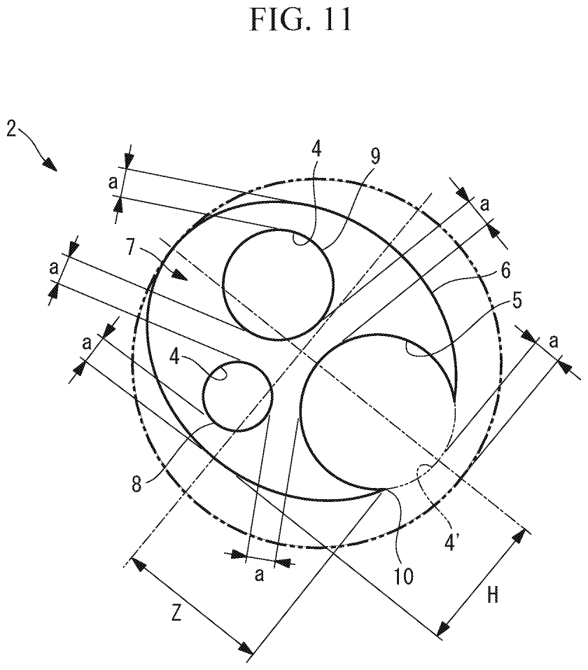

In addition, although the sheath 6 in which the light detecting portion 8 and the illuminating portion 9 are disposed in a single lumen 4 has been illustrated as an example in this embodiment, alternatively, as shown in FIG. 11, two lumens 4 may be provided in the sheath 6, and the light detecting portion 8 and the illuminating portion 9 may be separately disposed in the respective lumens 4.

In this case, the sheath 6 has a substantially elliptical cross-sectional shape satisfying the relationship H<Z, where Z is the radius of the sheath 6 in the major-axis direction, and H is the radius of the sheath 6 in the minor-axis direction.

The above-described embodiment leads to the following invention.

An aspect of the present invention is a blood-vessel optical device provided with: an elongated sheath provided with a lumen that penetrates therethrough in a longitudinal direction, and a dovetail groove that is disposed so as to be parallel to the lumen along the longitudinal direction; and an optical member that is accommodated in the lumen of the sheath, and that includes at least one of an illuminating portion that guides illumination light to an imaging subject in a blood vessel from outside of a body and a light detecting portion that guides return light from the imaging subject to outside of the body, wherein the dovetail groove has a cross-sectional shape configured to accommodate an elongated member having a circular cross section so as to be movable in the longitudinal direction, and has, on a side opposite to the lumen, an opening that has a width smaller than a diameter of the elongated member and from which a portion of the accommodated elongated member, in a circumferential direction, protrudes to the outside, and wherein the sheath is configured such that an external dimension in a direction orthogonal to an arrangement direction in which the lumen and the dovetail groove are arranged is smaller than an external dimension in the arrangement direction.

When the optical member is accommodated in the lumen and the elongated member having the circular cross section is accommodated in the dovetail groove, the portion of the elongated member, in the circumferential direction, is disposed in a state in which the portion protrudes to the outside from the opening of the dovetail groove. Because the opening of the dovetail groove has a width smaller than the diameter of the elongated member, the elongated member accommodated in the dovetail groove is maintained in the accommodated state without falling out from the opening. By configuring the portion for accommodating the elongated member so as to be movable in the longitudinal direction in the form of the dovetail groove, not in the form of the lumen, it is not necessary to ensure a sufficient thickness of the sheath at the position of the opening, and it is possible to reduce the external dimension in the arrangement direction in which the lumen and the dovetail groove are arranged. Furthermore, by setting the external dimension in the direction orthogonal to the arrangement direction in which the lumen and the dovetail groove are arranged to be smaller than the external dimension in the arrangement direction, it is possible to achieve a reduction in diameter of the sheath. In other words, by using a guide wire having a circular cross section as the elongated member, it is possible to facilitate introduction of the blood-vessel optical device into a thinner blood vessel while guiding the blood-vessel optical device with the guide wire.

In the abovementioned aspect, rounding-processing may be performed on a ridge of a boundary between an outer surface of the sheath and an inner surface of the dovetail groove.

By doing so, even if the external dimension of the sheath is minimized, and thus the ridge of the boundary between the outer surface of the sheath and the inner surface of the dovetail groove is formed to be thin, the rounding-processing can prevent sharpening of the ridge, thereby preventing the inner wall of a blood vessel from being damaged.

In addition, in the abovementioned aspect, the sheath may be composed of a material containing an X-ray contrast medium.

With this configuration, it is possible to clearly identify the blood-vessel optical device inserted into a blood vessel under X-ray observation, and to facilitate the insertion operation in the blood vessel.

In addition, in the abovementioned aspect, the optical member may be the illuminating portion, which is provided with an optical fiber scanner.

With this configuration, it is possible to radiate illumination light guided from outside of the body onto an imaging subject in a blood vessel in a state in which the blood-vessel optical device is introduced into the blood vessel.

In addition, in the abovementioned aspect, the optical member may be the light detecting portion, which is constituted of an optical fiber bundle.

With this configuration, it is possible to detect return light returning from an imaging subject by guiding the return light to outside of the body in a state in which the blood-vessel optical device is introduced into the blood vessel.

In addition, another aspect of the present invention is a blood-vessel optical system provided with: any one of the abovementioned blood-vessel optical devices; and the elongated member that is inserted into the dovetail groove of the blood-vessel optical device so as to be movable in the longitudinal direction.

In the abovementioned aspect, the elongated member may be a guide wire.

### REFERENCE SIGNS LIST

1 blood-vessel optical system
2 blood-vessel optical device
3 guide wire (elongated member)
4 lumen
5 dovetail groove
6 sheath
7 optical member
8 light detecting portion
9 illuminating portion
10 opening
X diseased site (imaging subject)

1. A blood-vessel optical device comprising:
an elongated sheath provided with a lumen that penetrates therethrough in a longitudinal direction, and a dovetail groove that is disposed so as to be parallel to the lumen along the longitudinal direction; and
an optical member that is accommodated in the lumen of the sheath, and that comprises at least one of an illuminating portion that guides illumination light to an imaging subject in a blood vessel from outside of a body and a light detecting portion that guides return light from the imaging subject to outside of the body,
wherein the dovetail groove has a cross-sectional shape configured to accommodate an elongated member having a circular cross section so as to be movable in the longitudinal direction, and has, on a side opposite to the lumen, an opening that has a width smaller than a diameter of the elongated member and from which a portion of the accommodated elongated member, in a circumferential direction, protrudes to the outside, and
wherein the sheath is configured such that an external dimension in a direction orthogonal to an arrangement direction in which the lumen and the dovetail groove are arranged is smaller than an external dimension in the arrangement direction.

2. The blood-vessel optical device according to claim 1, wherein rounding-processing is performed on a ridge of a boundary between an outer surface of the sheath and an inner surface of the dovetail groove.

3. The blood-vessel optical device according to claim 1, wherein the sheath is composed of a material containing an X-ray contrast medium.

4. The blood-vessel optical device according to claim 1, wherein the optical member is the illuminating portion, which is provided with an optical fiber scanner.

5. The blood-vessel optical device according to claim 1, wherein the optical member is the light detecting portion, which is constituted of an optical fiber bundle.

6. A blood-vessel optical system comprising:
the blood-vessel optical device according to claim 1; and
the elongated member that is inserted into the dovetail groove of the blood-vessel optical device so as to be movable in the longitudinal direction.

7. The blood-vessel optical system according to claim 6, wherein the elongated member is a guide wire.

8. The blood-vessel optical system according to claim 1, wherein the optical member comprises the illuminating portion, which is provided with an optical fiber scanner and the light detecting portion, which is constituted of an optical fiber bundle.
